(19) United States
(12) Patent Application Publication
(10) Pub. No.: US 2002/0142393 A1
(43) Pub. Date: Oct. 3, 2002

(54) Episomally replicating vector, its preparation and use

(76) Inventors: Armin Baiker, Lauffen/Neckar (DE); Jurgen Bode, Schoppenstedt (DE); Christian Fetzer, Munchen (DE); Hans-Joachim Lipps, Tubingen (DE); Christoph Piechaczek, Munster (DE)

Correspondence Address: Karen L. Elbing, Ph.D., Clark & Elbing LLP, 101 Federal Street, Boston, MA, 02110-2214, US

(21) Appl. No.: 10/059,492

(22) Filed: Jan. 29, 2002

Related U.S. Application Data
(63) Continuation-in-part of application No. 09/412,825, filed on Oct. 5, 1999, now Pat. No. 6,410,314.

(30) Foreign Application Priority Data
Oct. 17, 1998 (DE) 19848017.2

(51) Int. Cl. C12P 21/02; C12N 5/06; C12N 15/00; C12N 9/22

(52) U.S. Cl. 435/69.1; 435/199; 435/320.1; 435/325; 530/358

(57) ABSTRACT

The present invention relates to stably episomally replicating vectors, comprising at least one scaffold/matrix attached region (S/MAR) which binds to nuclear matrix proteins that contain a SAF-A consensus sequence, at least one viral or eukaryotic origin of replication (ORI), at least one transcription unit transcribed in direction towards the S/MAR, and a polyadonylation signal within the S/MAR or in transriptional direction after the S/MAR, cells comprising these, processes for their preparation, and their use, in particular as a medicament or diagnostic.

This is a continuation in part of U.S. application Ser. No. 09/412,825 filed Oct. 5, 1999.

The present invention relates to stably episomally replicating vectors, comprising at least one scaffold/matrix attached region (S/MAR) which binds to nuclear matrix proteins that contain a SAF-A consensus sequence, at least one viral or eukaryotic origin of replication (ORI), at least one transcription unit transcribed in direction towards the S/MAR, and a polyadenylation signal within the S/MAR or in transcriptional direction after the S/MAR, cells comprising these, processes for their preparation, and their use, in particular as a medicament

At present, vectors are widely used in research and therapy. In this context, vectors are used in particular for transforming or for transforming eukaryotic and prokarytic cells or cell systems and, in these, causing the expression of effectors such as pharmaceutically/medicinally relevant proteins or peptides, but also for replicating the vectors themselves. Effectors are understood in general as meaning substances which produce a particular effect of metabolic or therapeutic nature in the host cell. Customary effectors are nucleic acids coding for proteins or peptides, ribozymes, small RNAi's, antisense RNAs or are antisense DNAs.

Vectors are of particular importance in gene therapy. The fundamental object of gene therapy is the introduction of nucleic acids into cells in order to express an effector gene. Three fundamental problems exist here in gene therapy, a) the introduction of the gene (gene delivery), b) the maintenance of the gene (gene maintenance) and c) the expression of the gene (gene expression). In this context, just the maintenance of the gene and thus the stable and persistent expression of genes is a basic condition for successful gene therapy, which until now has not been solved very satisfactorily. The prerequisite for this is therefore the use of suitable vectors. In this context, in gene therapy in vitro and in vivo processes are differentiated in principle. In in vitro processes, cells are removed from the body and transfected ex vivo with vectors in order then to be introduced into the same or into another body again. In in vivo gene therapy, vectors are administered systemically. e.g. via the blood stream. However, local application, in which a gene-therapy vector is applied locally in the tissue, for example in an affected section of vessel, is also possible (see, for example, WO 95/27071)).

Thus, for the local application of a therapeutic gene in a selected case, for example, various strategies were developed based on modified balloon catheters, which are intended to permit direct administration of a substance or of a gene into the vascular wall. After a local administration using a double balloon catheter, Nabel, E. R. et al. (1990) Science, 249, 1285. for example, were able to detect a transient expression of the β-galactosidase gene in transfected cells of the femoral artery of the pig by means of liposomal and retroviral transfection.

Vectors are used in particular for the optimization of tissue-specific expression, which is used for the therapy of chronic diseases and hereditary diseases such as diabetes, hemophilia, ADA, muscular dystrophy, familial hyper-cholesterolemia or rheumatism, but can also be employed in acute diseases, such as vascular disorders—arteriosclerosis or its sequelae (stenosis, restenosis, cardiac infarcts)—and in tumors. Finally, the expression of genes and thus in particular the intercellular formation of therapeutic proteins and peptides, which on account of pathological or genetic modification are not or are no longer present to an adequate extent in the target organism, e.g. insulin or, in vascular cells, factor VII, etc., can also take place by means of a tissue-directed gene transfer.

An essential aim of somatic gene therapy is therefore to incorporate a therapeutic gene specifically into the target cells of the body after systemic or local administration and to express the therapeutic gene in those cells, without at the same time, however, inducing a transformation of the target cell or an immune response.

Up to now, there are two classes of vectors available for the incorporation and expression of therapeutic genes in somatic cells the vital vectors, where a differentiation has to be made here between a) episomally replicating vectors and b) vectors integrating into the DNA, and the nonviral vectors, in which c) a stable Transfection is achieved by random insertion (integrating) or d) (transient) only a temporary transfection is present The random integration into the host genome in approaches using integrating vectors can, depending on the integration point, lead both to insertion mutagenesis and to so-called “silencing”, in which no reading or expression of the inserted gene takes place. Transient extraction vectors are limited in their life in terms of time, not stable and in some cases also subject to integration, but sometimes also transform the host cell. Their most important disadvantage however, is that they often have to be repeatedly used on account of the limited expression associated with their short-lived nature. These vectors thus cause considerable problems just with respect to the effectiveness, reproducibility and safety necessary in somatic gene therapy.

The group of viral, episomally replicating vectors does not have these disadvantages, as these vectors are not integrated into the host genome and are retained in self-replicating form in the host cell. The term episomally replicating is understood here as me tat the vector is not integrated to the genome of the host cell, but exists in parallel, is also replicated during the cell cycle and in the course of this the vector copies—depending on the number of the copies present before and after cell division—are distributed statistically in the resulting cells. Plasmid vectors, for example the pGFP-C1 vector (Clontech UK Ltd), which have been optimized for research and other application purposes by alterations, are derived from the viral vectors. At present, only a few vectors are known which—starting from viral origins—replicate episomally in a few eukaryotic cells, e.g. SV40, BPV or EBV vectors. The replication origin of these vector. However, require interaction with one or more virally encoded reacting factors. These factors are also necessary for the stability of the vectors, but often lead to immortalization and transformation of the host cell or induce an immune response in the body (Ascenzioni et al. (1997) Cancer Letters 118, 135-142).

The eukaryotic virus SV40 (simian virus) thus replicates episomally in monkey cells and in some mammalian cells and cell lines. For this, the virus needs the so-called “large T antigen” for its existence in the host cell. The functions of the “large T antigen” are of crucial importance for the replication of the virus in the cell. The “large T antigen” binds, inter alia, to the viral DNA in the region of the origin of replication, and initiates its replication there (Mohr et al. (1987) EMBO J. 6, 153-160). Beside these activities which are important for the virus, the “large T antigen”, however, also affects cellular functions. It is bound, inter alia, to proteins which are involved in the regulation of the cell cycle (cyrlin, tubulins cdc2). Infections with SV40 or transfections with vectors which carry genes coding for SV40 “large T antigen” can therefore lead to the immortalization of primary cells and induce tumor formation in animals (Fried, M. (1965) Proc. Natl. Acad. Sci. USA, 53, 486-491; Eckhart, W. (1969) Virology, 38, 120-125; Di Mayorca el al. (1969) Virology, 830, 126-133).

WO 98/27200 discloses a construct containing a human or mammalian replication origin cloned in a circular vector, which—without being integrated into the host genome—replicates episomally in human cells. Cossons N. et al. (1997) J. Cell. Biochem. 67, 439-450 describe vectors that contain a matrix attachment region MAR) and different mammalian replication origin cloned in a circular vector. However, the episomal replication can only be maintained by selection pressure with selective antibiotics (G418) and even then occurs only with limited effectiveness. In fact the stability per generation was only 80% under selective pressure. Therefore, no stable maintenance of the episomally replicating vector was observed. While the use of selective antibiotics like G418 is feasible for at least a limited maintenance in tissue culture experiments it is not applicable to an in vivo animal or human gene therapy approach because of the high toxicity of the used antibiotics.

The previously known vectors therefore on the whole have considerable disadvantages and are only of very limited suitability for gene transfer, in particular in mammalian cells. The object of the present invention was therefore to develop a vector which has the advantages of ably episomally replicating viral vectors, without being dependent on trans-acting viral factors or expressing viral protein, and thus essentially to avoid any type of cell transformation or immune response, and to achieve an improved maintenance of the gene compared with the prior art.

The present invention therefore relates to a stably episomally replicating vector comprising at least one scaffold/matrix attached region (S/MAR) which binds to nuclear matrix proteins that contain a SAF-A consensus sequence, at least one viral or eukaryotic origin of replication (ORI), at least one transcription unit transcribed in direction towards the S/MAR (Bode et al, 2001), and a polyadenylation signal within the S/MAR or in transcriptional direction after the S/MAR (Bode et al., 2001).

Scaffold/matrix attached regions are understood as meaning sequences of nucleic acids which can subdivide chromatin in eukaryotic chromosomes in discrete domains, in particular in topologically connected so-called loop domains, and thus have crucial importance for structure and function, in particular, of the eukaryotic chromosome (Luderus, M. E. et al. (1994) Mol. Cell Biol., 14, 6297-6305). The loop domains essentially contain all necessary cis-regulatory elements for the coordinated expression of the genes within a so-called “domain”. The domains are limited by sequences which accumulate specifically on the nuclear matrix or the nuclear structure (scaffold). These sequences are called S/MARs and are usually several hundred base pairs long and rich in adenosine and thymidine (70%). Although cloned SAR and MAR elements have common structural properties, until now no consensus sequence has been identified (Boulikas, T. (1993) J. Cell. Biochem. 42, 14-22) S/MAR elements can increase the expression of heterologous genes after genomic integration (Klebr, D. el al. (1991) Biochem. 30, 1264-1270). S/MARs are credited with importance in the topological coiling of DNA (Bode, J. et al., (1992) Science 2555, 195-197). S/MAR elements can be isolated and identified, on the one hand, by the characterization of DNA bound in vivo to the nuclear matrix, on tho other hand by the characterization of DNA fragments which can bind to DNA-fee nuclear matrix in vivo (Lewin, B. (1994) Genes V, Oxford University Press, 776-778; Mielke et al. (1990) Biochem. 29, 7475-7485). Examples of the identification and characterization are found in Bode J. et al., (1995) (Scaffold Matrix Attachment Regions (S/MAR): Structural properties creating transcriptionally active loci, Int. Rev. Cytol. 162A, 389ff., Academic Press, Orlando) and Bode J. et al. (1992; supra).

S/MARs of over 1 kb in size have been considered as chromatin domain borders that play a critical role in nuclear architecture and nuclear function (Bode at al., 2000). A number of proteins binding to S/MARs in vitro have been identified within the past years. These include ubiquitous nuclear proteins such as toposomerase II (Adacbi et al., 1989), lamin B1 (Luderus et al., 1992), SATB1 (Dickinson and Kohwi-Shigematsu 1995), HMG. I/Y (Zhao et al., 1993) and histone H1 (Izaurralde et al,, 1989, Baiker et al., 2000). Other highly conserved proteins that bind specifically to S/MARs via an evolutionary conserved protein domain include SAF-A and SAF-B (scaffold attachment factor-A and -B) which represent major proteins of the nuclear matrix and therefore are considered crucial for nuclear architecture in vivo. Binding of S/MARs to lamin and SAF-A can be distinguished by competition binding studies, since the binding of S/MAR to lamins, but not the binding of S/MAR to SAF-A, can be inhibited completively by ssDNA. These competition binding studies revealed that S/MARs bind strongly and to about the same extent (50% respectively) to these two nuclear proteins (Bode et at. 2000, Crit Rev. Eukaryot Gene Expr 10:73-90).

S/MARs binding to nuclear matrix proteins that contain a SAF-A consensus sequence are understood as S/MARs that bind to proteins which contain an evolutionary conserved protein domain—also designated SAF-Box—comprising the following preferred 31 amino acid motif (SAF-Box of human SAF-A, the especially highly conserved parts are highlighted): L(or M) K V S E L K(or R)E E L K K R R L S D K G L K A E L M E R L Q A A. Almost identical amino acid motifs are found in many species such as human, mouse, zebra fish, Drosophila melanogaster, and S. corevisiae. Secondary structure predictions and computer-assisted modeling suggest a two-helix structure resembling homeobox-structures (Kipp et al., 2000; Renz and Fackelmayer, 1996).

In the present application, the binding of a S/MAR containing vector to a matrix protein is shown for the first time. Chromosomal rearrangements cannot be excluded in experiments employing a nuclear fractionation protocol (Fey el al., 1986) and subsequent South-Western analysis. For this reason, in order to study the interaction of an episomally replicating vector according to this invention with components of the nuclear matrix, the nuclear matrix fraction was prepared after crosslinking with cis-DDP (cis-dimedichloroplatinium II), a reagent that crosslinks matrix proteins to endogenous S/MARs with high specificity (Ferraro et al., 1996). By South-Wester analysis it was demonstrated that pEPI-1, but not the comparable vector pGFPC-1 lacking the S/MAR region, has a very high affinity to the matrix protein SAF-A. These observations contribute to a mechanistic explanation for the observed mitotic stability of the episomally replicating vector constructs described in this invention. Since the S/MAR-free counterpart to pEPI-1, pGFPC-1, integrates randomly into the genome (Baiker et al., 2000, Piechaczck et al., 1999), the mitotic stability of the vector according to this invention can be explained by the binding of the S/MAR region to nuclear matrix proteins containing a SAF-A consonsus sequence.

The protein domain of SAF-A binding to SARs is evolutionary conserved and was described to specifically bind to SARs but to be not related to SAR binding motifs of other proteins (Kipp et al., 2000). This domain was first identified in human scaffold attachment factor A (SAF-A) but is also present in many other proteins ranging from yeast to human in origin, This finding strongly suggests that other S/MAR regions, apart from the one studied in the preferred version of the vector according to the present invention, are comparably functional for the construction of episomally replicating vectors. This is further indicated in a recent publication by Shimizu et al. (2001), where extrachromosomal replication was shown in vitro with vectors comprising a MAR and a mammalian ORI. From this and from the knowledge of the person skilled in the art, appropriate isolation possibilities and use of S/MARS for the construction of episomally replicating sectors according to this invention result

The expression “origin of replication” (ORI) is understood as meaning the general starting point or origin of replication in eukaryotic or procaryotic cells and viruses. These ORIs support the replication and forming the attachment points for various replicators.

Methods for the isolation of the ORI sequences from animal cells are known to the person skilled in the art and are described, for example, In a review article by DePamphilis, M. L. (1993) Annul Rev. Biochem. 62, 29-63. Typical methods are, for example, “nascent strand extrusion” (Kaufmann G et al. (1985) Mol. Cell. Biol., 5, 721-727) or “anticruciform immunoaffinity purification” (Bell, D. et al. (1991) Biochem. Biophys. Acta, 1089, 299-308).

In consequence, it was completely surprising that a vector in which only one or more S/MAR elements are connected to one or more eukaryotic or viral ORIs and one or more transcription units, is, on the one hand, not integrated into the genome, which would have normally been expected according to the prior art (Wegner el al., (1989) Nucleic Acids Research 17, 9909-9932), and is on the other hand, episomally replicated without being dependent on in-trans-acting factors (of viral origin) for its replication. In addition, it has surprisingly also turned out that the vector according to the invention is stable and it is retained without selection by antibiotics for up to over approximately 100 generations. For these reason, the vectors according to the present invention are advantageous vehicles for gene therapy, research and all sorts of other application areas. Stable episomal replication within the present invention means, that the vector is retained in the transfected cell over at least 30 generations, preferably over at least 50 generations, more preferably over at least 80 generations, at least 100 generations, or at least 200 generations without the ongoing application of selective pressure. A vector is considered to be successfully retained if it can still be detected by Southern blot analysis and/or only a small number of cells die in tissue culture after re-addition of selective pressure (for instance G418). The vector according to the invention thus has, on the one hand, the advantage that the problem associated with random integration do not occur. On the other hand, as a result of the stable episomal replication a long-lasting action can be achieved, such that repeated treatment is not necessary, i.e. the problem of the gene retention in the transformed cell (gene maintenance) is essentially solved. Otherwise, without in-transacting factors (of viral origin) whose sequences are also already present in the host genome in many, just immortalized cells, transformation or immortalization of the host cell or induction of the immune response by viral proteins is not to be feared. The expression system is also based exclusively on chromosomal elements. The vector according to the invention therefore offers the necessary effectiveness, reproducibility and safety.

All in all, the vector components in general work together functionally such that the S/MAR allows episomal replication without the vector being integrated into the host genome or replication factors foreign to the cell having to be added to the ORIs for this. The S/MARs to this extent replace the replication factors or provide for activity of endogenous replication factors. The expansion of an ORI with S/MAR at least guarantees its functionality in plasmids.

In particular, the vector according to the invention can be an expression vector. The expression vectors have the advantage that they can express genes of very different types in the host cell. Expression vectors are understood as meaning vectors in which one or more genes coding for one or more peptides or proteins each are under the control of host-specific gene-regulatory sequences. Within the meaning of this invention, these are vectors which are suitable for the (episomal) expression of one or more genes and in addition to the corresponding gene sequence or sequences additionally also have promoter, operator and terminator sequences for the transcription and the sequence of the ribosomal binding sites for the translation. Within the meaning of this invention, different ones expressed by an expression vector can each be controlled by a distinct promoter. Straight expression vectors are very suitable for gene therapy or the in vitro expression of various genes both in eukaryotes and in prokaryotes.

The vector according to the invention can otherwise also be distinguished in that it does not contain any nucleic acids coding for replication factors which act in trans. As mentioned above, particularly the in-trans-acting factors Normally in vectors previously known from the prior art with viral ORI which was necessary for a replication of the information encoded on the vector are disadvantageous. The particular advantage of this embodiment is therefore that here these replication factors can be dispensed with, in particular those which show an action in trans and at the same time bring about, for example, a change in the host cell.

The expression replication factor within the meaning of this invention is understood generally as meaning factors which are necessary for the replication of the vector, that is, for example bind to the ORIs and bring about a doubling of the nucleic acids. Such replication factors can be both protein, and peptides. The expression the trans action mentioned in this connection is broadly interpreted within the meaning of this invention. Trans action is any action of a replication factor which is not immediately directed at the relatively close environment of its sequence coding for it. Examples of replication factors within the meaning of this invention would be the SV40 large T antigen, trans-activating factors such as EBNA1 from EBV vectors and E1 and E2 of the BPV vectors.

In a further embodiment, the vector does not contain any nucleic acids coding for replication factors—in particular also those of viral origin—completely independent of whether they act in-trans or not. This is possible, since here there is no longer any functional dependence on viral replication factors and the danger of the transformation is also better excluded.

A vector is particularly preferred which does not contain any nucleic acid coding for viral proteins at all. The advantage of this embodiment is that no viral proteins whatsoever are expressed any longer and thus the otherwise frequently occurring induction of the immune response is completely suppressed, which makes this embodiment very particularly suitable for therapy. An example of a viral protein which is simultaneously a replication factor and acts in-trans is the known “large T antigen” of the SV40 virus, which is known in its tumor-inducing or immortalizing action.

The present invention further relates to a vector in which the “origin of replication (ORI)” is used for propagation in eukaryotes, it preferably being selected from the group of the viral ORIs such as EBV-ORI. BPV-ORI or in particular SV40-ORI. Propagation in eukaryotes is used, in particular, in therapeutic applications and for research purposes in this field of application.

A vector according to the invention in which the ORI is used for propagation in prokaryotes, in this case preferably the pUC-ORI, likewise comes under the invention. A vector equipped in this way has the advantage that it can be utilized for the replication of the vector in prokaryotes and thus can be replicated comparatively simply in high yields.

In a particularly preferred embodiment of the vector, one or more “origin of replications” will be contained for propagation in eukaryotes and one or more for propagation in prokaryotes, preferably at least one for propagation in the eukaryote and at least one for propagation in the prokaryote. The advantage of this embodiment is that, on the one hand, the vector can easily be replicated in prokaryotes and, on the other hand, the same vector can be stably maintained in eukaryotes.

Vectors according to the invention are also those wherein the S/MAR originates from a mammal and is preferably even of human origin. The advantage of this embodiment is that particularly good propagation in eukaryotes can be achieved thereby, in particular in the course of gene therapy. A particularly preferred S/MAR here is that selected from the 5′ region of the interferon β gene of human origin, isolated as the 2.0 kb EcoRI/BglII fragment from the plasmid pTZ-E20, spanning nucleotides 217-2206 of SEQ ID No.1 (Bode el at. (1992) supra: FIG. 4 and Sequence protocol). In accordance with the finding, that the binding of S/MAR regions to nuclear matrix proteins that contain a SAF-A consensus sequence, promotes the mitotic stability of The episomally replicating vectors according to this invention, a further preferred embodiment are vectors including any other S/MAR region of mammalian, preferably human origin.

Episomally replicating vectors can also additionally containing one or more genes mediating antibiotic resistance, These are used, in particular, for selection and for control, whether a successful transfection or transformation of the cells treated with the vector is present. In this case, genes which mediate a resistance against antibiotics selected from kanamycin, geneticin, gentamycin, ampicillin, tetracycline, streptomycin, spectinomycin, nalidixic acid, rifamproin, chloramphenicol and/or zeocin are particularly preferred, since these antibiotics known to the person skilled in the art are suitable for the selection, it being possible to add to these any others from his expert knowledge.

A particularly preferred embodiment of the vector contains the SV40 ORI and a scaffold/matrix attached region sequence from the 5′ region of the interferon β gene, isolated as the 2.0 kbEcoRI/BglII fragment from the plasmid pTZ-E20(Bode et at. (1992), supra; FIG. 4). Prokarytic ORIs, such as the pUC ORI, genes mediating resistance, in particular against kanamycin, and various effectors can be added. A suitable starting vector which would be modified by the insertion of the various above mentioned regions to give a vector according to the invention would be the pGFP-C1 vector of the company Clontech UK Ltd. (see FIG. 2).

In a further example, the vector according to the invention is distinguished in that it contains one or more promoter or activator sequences and/or one or more effectors.

Promoters are understood as meaning nucleic acid sequences which usually lie 5′ from the sequence to be read and regulate the transcription rate of a gene. A differentiation is made here between activator and repressor sequences, which respectively increase or decrease the gene activity. “Enhancers” can be counted among the activators and differ from other regulation elements in that they lie at a greater distance from the 5′ or 3′ and can increase the transcription activity in a position-independent manner, e.g. from human cytomegalo-virus (EP 0 173 177), CMV immediate-early polypepide (Pos. 216-809/Genbank Accession No.: K03104).

Particular groups of activator sequences and promoters which are also preferred here are constitutive, cell cycle-specific, tissue-specific, metabolically regulated and/or inducible promoters or activator sequences. On the whole, these have the advantage, depending on choice, of being appropriate to the cell situation, so that particular metabolic conditions or therapeutic needs of a cell can be taken into account or the replication or expression can be controlled by external factors.

Preferred effectors code for certain substances, selected from proteins, peptides, ribozymes, small RNAi's, antisense RNAs, or air antiscose DNAs. Peptides are understood here as meaning a part of a protein, or an amino acid sequence, either of natural or synthetic type. The function of these vectors is extremely diverse and can be tailored to the particular therapeutic needs. In the widely diversified literature, many examples of this are available, coding sequences being known, in particular for therapeutic proteins. Without restricting the application possibilities of the vector according to the invention thereto, or this listing being intended to be complete, a few examples are mentioned here in which proteins, or genes coding for these proteins, can be used therapeutically in this connection: nitrogen monoxide synthase (see, for example, WO 95/27020), insulin (see, for example, EP-B 100 01 929), erythropoietin (see, for example, EP-B3-0 148 605), or blood clotting factors, such as, for example, factor VII, inferferons, cytokines, hormones, growth factors etc. The choice of the suitable effectors employed in the vector remains left to the knowledge of the person skilled in the art.

A further subject of the present invention is also one or more cells which contain one or more of the vectors described above. Thus, embodiments of the invention are in particular described in which, for the storage or propagation of the vector, tis is already included in a cell. Particularly preferred here are eu- or prokaryotic cells, in particular bacterial, yeast, insect, amphibian, fish or mammalian cells. In this case, it is, for example, also known in the case of fish cells that expression occurs after microinjection of foreign DNA (Winkler et al. (1991) Mol. Gen. Genet. 226, 129-140). Transgenic fish can likewise be produced (WO 96/03034; WO 96/32087; WO 98/15627).

Especially in gene therapy, nonimmortalized cells of human origin are preferred. The term “nonimmortalized” is to be understood in this connection as meaning that the cell is not transformed in the genome, i.e. not replicable at will, but is subject to the natural cell cycle and thus—in contrast to the tumor cell is itself of limited life span and can only replicate without a limited framework (Alberts et al., Molecular Biology of the Cell: Cancer (1995) 3^{rd} Ed.). Examples for the successful propagation of the stably episomally replicating vector according to this invention are the use of primary human keratinocytes (HaCat cells, see examples 6 and 7), human hepatocytes, and primary human myoblasts (Campeau et al., Abstact 2000).

A further embodiment of the invention is transgenic, preferably embryonic, stem cells, which contain the vector according to the invention and/or nucleic acids produced therefrom and, for example, allow the production of transgenic animals, as well as The transgenic animals themselves, in which some or all cells of the animal contain the vectors according to the invention nucleic acids produced therefrom and/or, if appropriate, expression produces or the genes (see WO 90/03432, WO 95/06716, EP 0 169 672, DE 196 32 532, WO 96/03034; WO 96/32087; WO 98/15627).

A further subject of the present invention is also a process for the preparation of a vector according to the invention, in which one or more scaffold/matrix attached regions, that bind to nuclear matrix proteins that contain a SAF-A consensus sequence, are combined with at least one ORI and at least one transcription unit transcribed in direction towards the S/MAR (Bode el al., 2001) but no nucleic acids for SV40 T antigen. The best-known method for the preparation is the separation of a region from plasmids or other nucleic acids and the insertion or ligation into a vector, plasmid or other nucleic acid with the aid of restriction endonucleases (restriction enzymes).

A particular form of the process consists in replacing one or more of the nucleic acids coding for replication factors in the original vector by at least one S/MAR region which binds to nuclear matrix proteins that contain a SAF-A consensus sequence, This is carried out by existing these regions by means of restriction enzymes and inserting one or more S/MAR fragment(s) into the vector using the methods known to tho person skilled in the art.

In addition, this particular form of the process including the insertion of at least one viral or eukaryotic ORI and/or a gene mediating antibiotic resistance. It is further necessary and useful in many application areas to insert into the vector at least one transcription unit transcribed in direction towards the S/MAR (Bode et al., 2001), preferably coding for a peptide or a protein. A transcription unit is generally understood as the region of a nucleic acid molecule between sites of initiation and termination of the transcription of a gene by RNA polymerase.

In this particular form of the process it is further useful to insert a polyadenylation signal within or in transcriptional direction after the S/MAR (Bode et al., 2001). A polyadenylation signal is understood as a shorn consensus sequence such as TTATTT that, transcribed into mRNAs of higher eukaryotes, leads to cleavage of the RNA 3′ end and subsequent addition of a poly-A sequence. Examples for polyadenylation signals according to this invention are those isolated from HSV-tk (Herpes simplex virus thymidine kinase) or SV40. This polyadenylation signal within or after the S/MAR causes the RNA polymerase II to partially transcribe through the S/MAR element.

A further embodiment of the process described in the paragraph above is the additional insertion of multiple genes, each under the control of a distinct promoter

There are numerous applications for the vectors or cells according to the invention, for example the transfer of substances, in particular of pharmaceutically active compounds, especially for gene transfer. Gene transfer is used, for example, for the diagnosis or therapy of vascular and/or organ disorders. Gene therapy is of particular importance here. In this case, the genes integrated into the vectors are expressed in the target cell—for example by the action of an expression vector. This applies in particular to genes which code for pharmaceutically and medicinally relevant proteins. In particular, the episomally replicating vector according to the invention allows a particularly side effect-free use in the therapeutic respect and a particularly preferred use is that as a “shuttle vector” in gene therapy. A “shuttle vector” is understood as meaning a vector which can be propagated in at least two different cell types, or organisms, for example vectors which are first propagated or replicated in prokaryotes in order for, for example, eukaryotic cells then to be able to be transfected with these.

The in vitro expression of one or more genes is likewise important as a use of the vector according to the invention or its cells. The vector thus makes possible a strong expression of genes and thus, for example, the preparation of proteins and peptides in large amounts in various cells and cell systems of both eukaryotic and prokaryotic type, without continuously placing the cells under selection pressure, which adversely effects both the protein yield and increases the process costs. Using the vector, it is also possible to express genes which code for proteins or peptides and which until now it has not yet been possible to express without difficulty—in particular in sensitive cell systems.

A further aspect of the invention is also the use of a vector according to the invention for the transfection of cells. Transfection is understood as meaning the inclusion of the vector in the cell. Thus, on the one hand, the transfection step necessary in gene therapy is meant, as well as the transformation of prokaryotic cells, for example for the propagation of the vector.

Otherwise, the invention also includes the use of the vectors according to the invention for the production of transgenic animals or stem cells, for example embryonic stem cells, since these vectors are suitable for use, in particular, in eukaryotic cells and also for use for research purposes. Transgenic animals are to be understood as meaning those in whose cells the vectors according to the invention and, if appropriate, effectors propagated thereby are present. Transgenic stem cells are understood as meaning cells which are transfected using the vectors according to the invention and from which, for example, transgenic animals can be produced or reared. Examples are disclosed in WO 96/03034, WO 96/32087, WO 98/15627, WO 90/03432, WO 95/06716, EP 169 672 and DE 19 632 532.

The design of non-viral episomal vectors will be the solution of choice for the efficient and reproducible modification of eukaryotic cells and organism and eventually also for the safe therapy of human diseases.

The invention in this case also includes as a further subject a composition which contains at least one of the vectors according to the invention and/or a cell which contains such a vector, and suitable additives and/or auxiliaries.

The suitable additives and auxiliaries are to be understood as meaning, in particular, adjuvants, stabilizers and/or transfection-facilitating substances. Also covered are transfection systems including transfection vectors, which are combined or associated with the vector according to the invention and its penetration into cells, which facilitate or even allow transfection or alternatively transformation. Auxiliaries are in particular to be understood as also meaning general protease inhibitors, such as PMSF, and nuclease inhibitors, such as EDTA.

Preferred transfection vectors are, for example, viral or nonviral vectors. It is further possible to use for the transfection other, nonviral, transfection-facilitating substances, for example those from a lipid, a polymer, a peptide or a porphyrin, also in combination with vectors.

Gene-therapy vectors can be obtained by complexing the vector according to the invention with liposomes (neutral or cationic). The vector is thus essentially included in the liposome, thus has a very high transfection efficiency (see, for example, WO 95/27070) and is essentially protected from DNAses. Transfection with nucleic acid-liposome complexes with the aid of Sendai viruses in the form of so-called HVJ liposomes (virosomes) is particularly advantageous, as by this means the transfection rate can be increased still further.

During lipofection, small unilamellar vesicles are prepared from cationic lipids by ultrasonic treatment of the liposome suspension. The vector is bound ionically to the surface of the liposomes, to be precise, for example, in such a ratio that a positive net charge remains and 100 percent of the vector is complexed by the liposomes. In addition to the lipid mixture DOTMA (1,2-dioleyloxylpropyl-3-trimetbylammonium bromide) and DOPE (dioleylphosphatidylethanolamine) compound by Felgner et al., (Felgner, P. L. et al (1987) Proc. Natl. Acad Sci. USA 84, 7413-7414), in the meantime numerous novel lipid formulations have been synthesized and tested for their efficiency on the transfection of various cells. Examples of the novel lipid formulations are DOTAP or DOGS. An example of the preparation of DNA-liposome complexes from phorsphatidylcholine, phosphatidylserine and cholesterol and their successful use in the transfection of vascular walls with tho aid of Sendai viruses is described in WO 95/27070.

It is particularly advantageous if the vector-liposome complex contains nucleic acid-binding proteins, for example chromosomal proteins, preferably HMG proteins (high mobility group proteins), in particular HMG1 or HMG2 or nucleosomal histones, such as H2A, H2B3 or H3 or H4, since by this means the expression of the gene integrated in the vector can be increased. The chromosomal proteins can be isolated, for example, from calf thymus or rat liver according to generally known processes or prepared by genetic engineering. Human HMG1 can for example, be prepared particularly easily recombinantly by methods known to the person skilled in the using the human cDNA sequence (Wenn, L. et al. (1989) Nucleic Acids Research 17(3), 1197-1214).

A histidine-containing polypepide which increases membrane permeation can likewise be employed. A so-called polyfection solution, comprising a vector according to the invention with the desired effector, a fusion protein made from tissue-specific peptide and a DNA-forming portion, e.g. a positively charged domain, and a peptide which increases membrane permeation, is preferably employed. In addition, coupling of the vector to the liposomes by means of a, for example, introduced C-terminal cysteine to an activated lipid component is known

A further subject of the present invention is a medicament or a diagnostic which comprises an episomally replicating vector having at least one scaffold/matrix attached region and at least one viral or eukaryotic origin of replication and/or one or more of these vector-containing cells and, if appropriate, suitable additives or auxiliaries (see above).

Another embodiment of the present invention also relate to a composition, for example in the form of a transfection system, comprising one or more vectors and/or cells comprising these vectors and a further substance, for example for the transfection of cells. The polyfection solution described above would be particularly preferred here.

The following figures and examples are intended to describe the invention in greater detail without restricting it:

### FIGURES

FIG. 1 shows a vector with the sketched regions present on this vector as an exemplary embodiment

In this particularly preferred embodiment, the following sequence elements are found; an SV40 ORI (135 base pairs) for propagation in eukaryotes, a kanamycin resistance gene (1399 base pairs) for selection both in E.coli and in eukaryotes (mediates resistances to kanamycin or geneticin), a pUC-plasmid ORI (643 base pairs) for propagation in E.coli and a matrix attached region (from the 5′ region of the human interferon 13 gene, 1984 base pairs) for propagation in eukaryotes.

On interaction of these elements with, for example, an effector element, by means of the cooperation of the matrix attached region, in particular with the SV40 ORI, an episomally replicating vector results whose transfection can be checked by the kanamycin resistance gene and which propagates in prokaryotes through the pUC-ORI and can thus be prepared in an adequate amount.

FIG. 2 shows the pGFF-C1 vector employed according to Examples 1 and 2, as was supplied by the company Clontech and which was used in the examples and a preferred preparation process.

FIG. 3 allows a particular embodiment of the vector according to the invention, designated here as pEPI-1, using which some of the examples were carted out.

According to Example 1, S/MAR was integrated here into a vector according to FIG. 2, so that a vector according to the invention results. This contains an (S/)MAR, a 2.0 kbEcoRI/BglII fragment of the plasmid pTZ-E20 from the 5′ region of the interferon β gene according to FIG. 4, the SV40 ORI, the pUC ORI, the resistance gene Kan/NGO with associated HSV TK poly A and promoter pₐₘₚ, the “enhancer” pCMV, the “SV40 early promoter” pSV40 and the GFP/green fluorescent protein. The results of Examples 1 to 5 have also been achieved using an appropriate vector.

FIG. 4 shows the plasmid pTZ-E20.

SEQ ID NO:1 shows the nucleic acid sequence of the human interferon β S/MAR

### EXAMPLES

### Example 1

### Preparation of a Preferred Episomally Replicating Vector

An S/MAR fragment from the 5′ region of the human interferon β gene was isolated from the plasmid pTZ-E20 (Bode, J. et al., loc. cit.) as a 2.0 kbEcoRl/BglII fragment and inserted into the polylinker PGFP-C1 (see FIG. 2). A vector according to the invention, designated as pECPI-1, resulted thereby, In another experiment, the gene coding for the SV40 “large T antigen” was excised from another viral/plasmid vector and replaced by S/MAR and a vector according to the invention was thus also obtained.

### Example 2

### Transfection and Selection of Eu- and Prokaryotic Cells

Chinese hamster ovary (CHO) cells were cultured in Ham's F12 medium with 10% FCS, 2.5 μg/ml of amphoteriein B and 50 μg/ml of gentamycin. 3×10⁶ CHO cells were electroporated and incubated with 5 μg of the vector pEPI-1, prepared according to Example 1, (FIG. 3) or pGEP-C1 (FIG. 2) One day after the electroporation, transfected cells were selected by means of 500 μg/ml of G418, transfected cells surviving on account of their antibiotic resistance. After two weeks, stable clones were isolated and cultured with 250 μg/ml of G418. A similar procedure was used with E.coli cells.

### Example 3

### Retransfection

A HIRT extract (Hirt, B. (1967) J. Mol. Biol., 26, 365-369) obtained from the transfected CHO cells according to Example 2 was used in order to transfect new CHO cells according to the procedure in Example 2.

### Example 4

### Results of Investigations of the Cells According to Examples 1-3

After isolation of the DNA, digestion with restriction enzymes, blotting and hybridization experiments with a labeled pEPI-1 probe, it was found that random integration of the vector had not taken place in Example 2 and to be precise in any of the clones. The vector according to the invention. In this case pEPI-1, did not show any hybridization with the chromosomal DNA, while a HIRT extract obtained from cells according to Example 2 and Example 3 showed an isolated DNA with the restriction pattern identical to the vector according to the invention. By transformation of the isolated episomal DNA in CHO cells, it was possible to detect the vector (see Example 3), as well as in E.coli cells, This contradicts the result known in the prior am that in a highly amplified vector which has carried an AT-rich sequence of another type a head-to-tail integration takes place (Wegner et al. (1989) Nucleic Acids Research 17, 9909-9932). However, vectors which carry only a corresponding ORI or only S/MAR integrate randomly into the genome of the host (see also Klehr el al., (1992) Biochemistry, 31, 3222-3229, and Schübeler et al., (1996) Biochemistry, 35, 11160-11169). Thus, it is incidentally also demonstrated that CHO cells express no T antigen since otherwise no integration of the vectors only carrying ORIs would take place. Furthermore, the results of a Southern analysis have also shown that the vectors according to the invention replicate efficiently and stably extrachromosonally; they are thus episomal vectors, about 20 copies of the vector being present in each clone.

### Example 5

### Stability and Expression Investigations

In order to investigate the plasmid stability and the expression of the neomyein resistance gene, transfected CHO cells according to Example 2 were cultured for more than 2 months (at least 100 generations) in a medium according to Example 2, but without addition of G418 and therefore without selection pressure by antibiotics.

If at different times during the entire culturing period since of the cultured G418 cells were added to the medium, only an insignificant number died in each case. It was also possible by means of Southern analysis to detect the episomal vector separately at any time.

It can be concluded, however, from this that on the one hand the vector is stable in CHO cells even without selection over at least 100 generations and on the other hand also the kanamycin resistance and thus a nucleic acid sequence inserted in the vector in the form of an effector, is expressed in each generation.

### Example 6

### Propagation of the Vector in Human Cells

HaCat cells (human skin keratinocytes) were cultured in DMEM (Dulbecco's modified Eagle Medium) with 10% FCS. The HaCat cells were—in the same manner and under the same conditions as described in example 2—transfected with vector PEPI-1 prepared according to example 1 and selected. 4 weeks after the beginning of the selection stable clones were isolated and cultured with 250 μg/ml of G418.

### Example 7

### Results of Investigations of the Cells According to Example 6

The DNA of 6 clones according to example 6 was isolated. A Southern analysis as described in example 4 was conducted and in a further experiment the whole vector was amplificated by PCR. Two primers in opposite facing were selected from the Neo Gene (neo-fwd and neo-up), resulting in only circular molecules being amplificated. Both experiments showed that the vectors according to the invention replicate efficiently and stably extrachromosonally; they are thus episomal vectors, about 20 copies of the vector being present in each clone. The Neomyein-cassette was efficiently expressed. The results show that vectors according to the invention can be propagated and expressed episomally in human cells as well.

### Example 8

### Suitability for Ex Vivo Therapy of Duchenne Muscular Dystrophy

Primary human myoblast were successfully transfected with a preferred form of the vector according to this invention, pEPI-1. The vector was stably replicated in human primary fibroblasts for more than two months. This suitability of the episomally replicating vectors according to the present invention for the stable transfection of primary human myoblasts allows their use for ex vivo gene therapy e.g. by transfection myoblasts of patients with Duchenne muscular dystrophy with the dystrophin gene.

### Example 9

### Binding of the Vector to SAF-A in Vivo

### Cells and Vectors

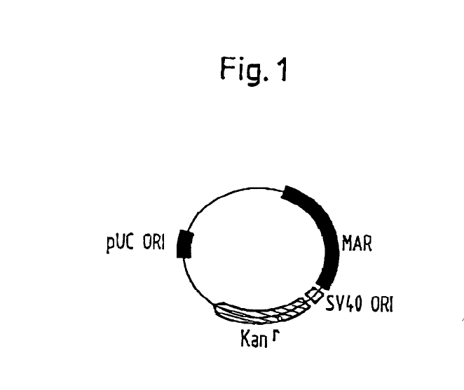

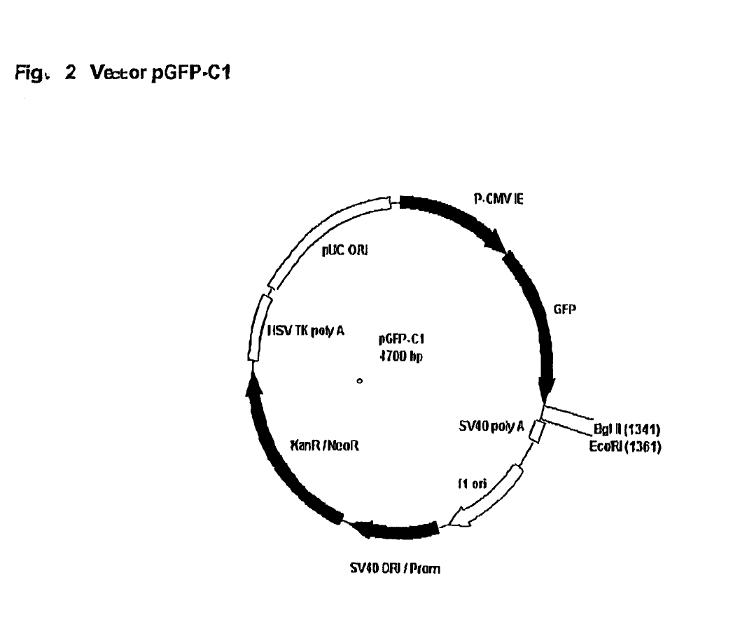

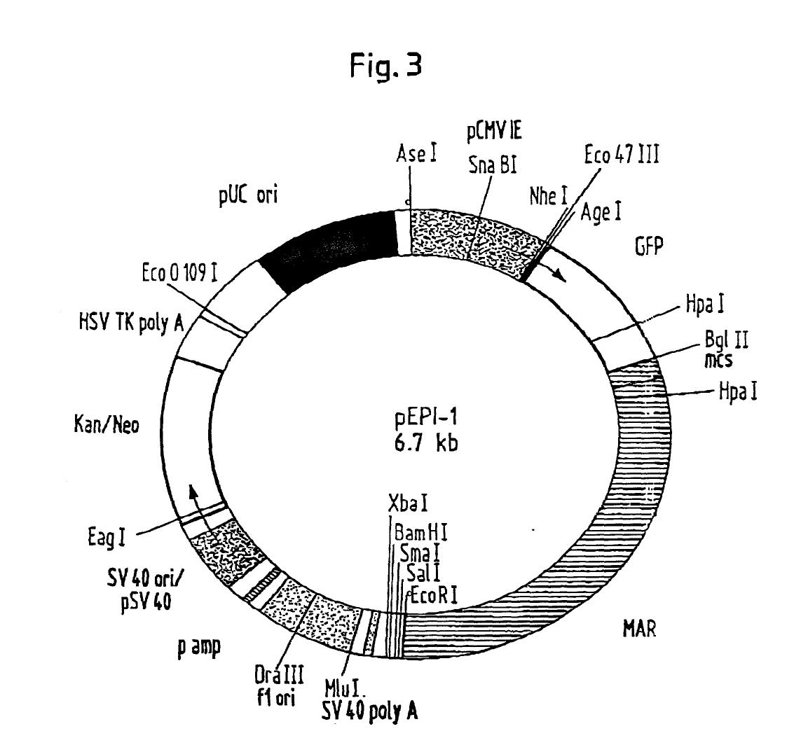

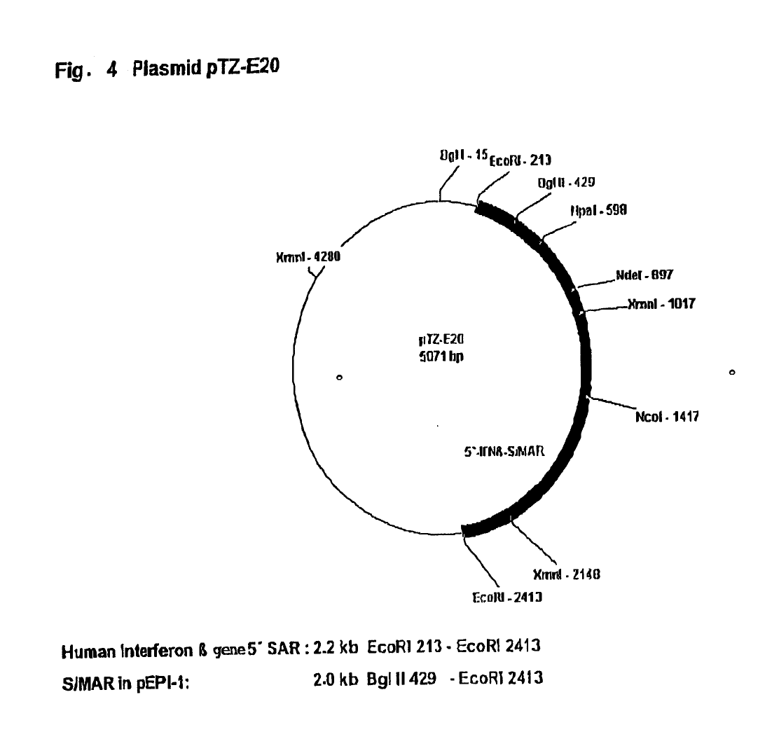

CHO cells were grown, transfected and selected as described earlier. Vectors used were pEPI-1 (Piechaczek et al., 1999) and pGFP-C1 (Clontech, FIG. 1a), while pEPI-1 replicates episomally in the absence of selection pressure in CHO cells, its S/MAR free counterpart pGPP-C1 consistently integrates into the genome in a random fashion (Piechaczek at al., 1999)

### Nuclear Fractionation

Nuclear fractionation followed the procedure described by Fey el al (1986). In this procedure, nuclear proteins are fractionated according to their resistance to salt treatment, resulting in a matrix preparation in the last fraction. Cells were washed with PBS and centrifuged, Cells were extracted in cytoskeletal buffer (10 mM PIPES, pH 6.8, 300 mM sucrose, 100 mM NaCl, 3 mM MgCl₂, 1 mM EGTA) containing 0.5% Triton X-100. In this step, soluble proteins, both cytoplasmic and nucleoplasmic are removed. Cells were extracted with extraction buffer (10 mM PIPES, pH 6.8, 250 mM ammonium sulfate, 300 mM sacrose, 3 mM Mg Cl₁, 1 mM EGTA), removing histone H1 and stripping the cytoskeleton except for the intermediate filaments that remain tightly anchored to the nuclear lamina. Chromatin was removed by discretion with RNase-free Dnase 1 in digestion buffer (10 mM PIPES, pH 6.8, 300 mM sucrose, 50 mM NaCl, 3 mm MgCl₂, 1 mM EGTA) containing 0.5% Triton X-100. This step removed DNA and the remaining histones. Left was the structure of the complete nuclear matrix. This structure was extracted in 2 M NaCl buffer (10 mM PIPS, pH 6.8, 300 mM sucrose, 2 M NaCl, 3 mM MgCl₂, 1 mM EGTA) stripping some proteins from the nuclear matrix and leaving the core structure of the nuclear matrix.

### Crosslinking with cis-DDP

Cis-DDP is a reagent that links matrix proteins to S/MARs with high specificity in low [Cl⁻] media (Ferraro et al., 1996), thus preventing chromosomal rearrangements during matrix preparation. 1×10⁸ cells were rinsed in 30 ml Hanks buffer with 300 μl 100 mM PMSF. Cells were collected by certification and washed. The pellet was resuspended in 10 ml 1 mM cis-D)DP-(cis-diamminedichlorophatinum).-solution and incubated for 2 hr at 37° C. (Ferraro et al., 1992). Crosslinking is immediately stopped by access of [Cl⁻] during the steps performed below.

### Isolation of Proteins After Crosslinking (Ferrara et al., 1992)

Following the crosslinking step, the cells were collected and resuspended in 80 ml of lysis buffer (5 M urea, 2 M guanidine hydrochloride, 2 M NaCl, 1 mM PMSF). The lysate was added to 1.6 g hydroxyapatite (HAP, BioRad) preequilibiated with lysis buffer and incubated on orbitron for 1 h at 4° C. The pellet was washed 3 times with ice cold lysis buffer. The crosslink between protein and HAP-bound DNA was reversed by the addition of 80 ml ice cold reverse lysis buffer (1 M thiourea, 2 M idine hydrochloride, 2 M NaCl, 1 mM PMSF) and incubated on orbitron for 2 h at 4° C. After certification for 30 min at 6.200 g. DNA-bound proteins could be recovered from the supernatant. This supernatant was dialyzed against distilled water containing 1 mM PMSF for 24 h, 4° C. and concentrated by certification through Vivaspin 20 (Sartorius, CO 10 000).

### Isolation of DNA After Crosslinking (Ferraro et al. 1992)

Following crosslinking the DNA was either fragmented by sonification as described earlier or by restriction digestion. In the latter case crosslinked cells were collected by certification, resuspended in 1 ml TE and digested 2 h at 37° C. with 5 restriction enzymes not cutting the vectors (200 U each, Eco RV, Pvi I, Xho I, Not I, Cla I)) and one enzyme (Eco RI, 200 U) linearizing them. The cells were then collected by certification, resuspended in 80 ml lysis buffer and bound to HAP as described above. Dissociation of DNA and DNA/protein complexes from HAP was achieved by incubation in 80 ml 0.4 M sodium phosphate buffer pH 7.2 on orbitron for 2 h at 4° C. After centifugation for 30 min at 6.200 g at 4° C. the supernatant was dialyzed against TE for 20 h at 4° C. To precipitate protein containing complexes, SDS was added to a final concentration of 1%, incubated 10 min at 37° C., then KCl was added to a final concentration of 0.1 M, incubated for 10 min on ice and centrifuged for 10 min at 3.000 g at 4° C. The pellet was resuspended in 20 ml TB containing 0.1 M KCl and incubated for 10 min at 65° C. followed by 10 min incubation on ice and centrifugation for 10 min at 3.000 g. This step was repeated twice, the precipitate was resuspended in 5 ml 10 mM Tris, pH 8.0, 10 mM EDTA, 100 mM NaCl, 0.4 mg proteinase K and incubated for 24 h at 37° C. DNA was extracted with phenol-chloroform and precipitated with ethanol. PCR Analysis

The presence of vector DNA in the various fractions and after immunoprecipitation was detected by PCR analysis PCR conditions and primers used were as described (Baiker et al., 2000).

### Immunoprecipitation

Immunoprecipitation of the HAP-bound DNA-protein complex with anti-SAF-A antibody (Fackelmayer and Richter, 1994) was done as follows: Following crosslinking lysates from about 10⁸ cells were bound to HAP as described above. Binding was reversed by incubation in 0.4 M sodium phosphate buffer pH 7.2, HAP was removed by centrifugation and the supernatant dialyzed against TE and concentrated in Vivaspin 20 to a final volume of about 200 μl. 10×PBS to a final concentration of 1×PBS was added. 12 μl/ml anti-SAF A antibody was added and incubated on ice overnight. The resulting precipitate was collected by centrifugation for 2 h at 13.000 g at 4° C. and washed twice with PBS. The precipitate was resuspended in 40 μl TE and the DNA was isolated as described above. Aliquots of the DNA were used to amplify the vector DNA by PCR.

### Gel Electrophoresis, South-Western—and Western Analysis

DNA was separated on 1% agarose gels in TAB. Proteins were fractionated an 12.5% SDS-polyacrylanide gels (Laemmli, 1970) and transferred to nylon membranes by electroblotting. Western analysis was performed as described earlier (Towbin et al., 1979). using the anti-SAF A antibody (dilution 1:500) and the anti-rabbit IgG (Dako. Hamburg, dilution 1:1000) South-Western analysis followed the protocol of Mislms et al. Miskimis et al., 1985) with 0.2 μg DIG-labeled (Kessler et al., 1990) pEPI-1 or pGFPC-1. Binding with pEPI-1 was performed in the presence of a 10 fold excess of unlabeled pGFPC-1, binding of pGFPC-1 in the presence of a 10 fold excess of unlabeled pEPI-1.

It will be apparent to those skilled in the art that various modifications can be made to the compositions and processes of this invention. Thus, it is intended that the present invention cover such modifications and variations, provided they come within the scope of the appended claims and their equivalents.

Priority application DE 19848017.2, filed Oct. 17, 1998 and priority application U.S. Ser. No. 09/412,825 filed Oct. 5, 1999. All publications cited wherein are incorporated in their entirety by reference.

We claim:

1. A stably episomally replicating vector wherein said vector comprises at least one scaffold/matrix attached region (S/MAR) which binds to nuclear matrix proteins that contain a SAF-A consensus sequence, at least one viral eukaryotic origin of replication (ORI), at least one transcription unit transcribed in direction towards the S/MAR, and a polyadenylation signal within the S/MAR or in transcriptional direction after the S/MAR.

2. The vector of claim 1, wherein said S/MAR is encoded by a 20 kb EcoRI/BglII fragment spanning nucleotides 217-2206 of SEQ ID No. 1 coding for the S/MAR region from the interferon β gene.

3. The vector of claim 1, further containing an ORI for propagation in a prokaryote that is the pUC ORI.

4. The vector of claim 1, wherein said vector is retained in episomal form for over at least 30 generations of cell divisions without the ongoing application of selective pressure.

5. The vector of claim 1, wherein said vector is retained in episomal form for over at least 50 generations of cell divisions without the ongoing application of selective pressure.

6. The vector of claim 1, wherein said vector is retained in episomal form for over at least 80 generations of cell divisions without the ongoing application of selective pressure.

7. The vector of claim 1, wherein said vector is retained in episomal form for over at least 100 generations of cell divisions without the ongoing application of selective pressure.

8. The vector of claim 1, wherein said vector is retained in episomal form for over at least 200 generations of cell divisions without the ongoing application of selective pressure.

9. The vector of claim 1, wherein said vector further comprises at least one promoter selected from the group of promoters consisting of constitutive promoters, cell cycle-specific promoters, tissue-specific promoters, metabolically regulated promoters, and inducible promoters.

10. The vector of claim 1 wherein said vector further comprises an activator sequence selected from the group of activators consisting of constitutive activators, cell cycle-specific activators, tissue activators, metabolically regulated activators, and inducible activators.

11. The vector of claim 1, wherein said vector comprises more than one transcription unit transcribed in direction towards the S/MAR, each under the control of a distinct promoter.

12. The vector of claim 1, wherein said vector does not contain a nucleic acid coding for viral proteins.

13. The vector of claim 1, wherein said vector further comprises at least one gone mediating antibiotic resistance.

14. The vector of claim 13, wherein said gene mediates resistance to an antibiotic selected from the group of antibiotics consisting of kanamycin, geneticin, gentamicin, ampicillin, tetracycline, streptomycin, spectinomycin, nalidixic acid, rifampicin, chloramphenicol, and zeocin.

15. The vector of claim 1, wherein said vector further comprises a polynucleotide sequence coding for a substance selected from the group consisting of proteins, peptide, ribozymes, small RNAi's, and antisensor RNAs.

16. The vector of claim 1, wherein said expression vector contains a nucleic acid coding for a nitrogen monoxide synthase insulin, erythropoietin blood clotting factor, interferon, cytokine, hormone, or growth factor.

17. An isolated cell comprising the vector of claim 1.

18. The cell of claim 17, wherein said cell is a eukaryotic or prokaryotic cell.

19. The cell of claim 17, wherein said cell is selected from the group of cells consisting of a bacterial, yeast insect amphibian, fish, and mammalian cell.

20. The cell of claim 17, wherein said cell is a non-immortalized cell of human origin.

21. The cell of claim 20, wherein said cell is selected from the group consisting of human keratinocytes human hepatocytes, and primary human myoblasts.

22. A process for the preparation of a vector of claim 1, comprising the step of inserting one or more S/MARs which binds to nuclear matrix proteins that contain a SAF-A consensus sequence, in a vector containing at least one SV40 or BPV ORI, and at least one transcription unit in direction towards the S/MAR but no nucleic acids for SV40 T antigen.

23. A process for the preparation of a vector of claim 1, comprising replacing one or more nucleic acids coding for SV40 T antigen in a vector by at least one S/MAR binding to nuclear matrix proteins that contain a SAF-A consensus sequence.

24. The process of claim 22, wherein at least one ORI or a gene mediating antibiotic resistance is further inserted into said vector.

25. The process of claim 22, wherein at least one nucleic acid coding for a peptide or protein is further inserted into said vector.

26. The process of claim 22, wherein multiple genes, each under the control of a distinct promoter, are further instead into the vector.

27. A process for transfecting a cell, said process comprising contacting said cell with a vector of claim 1.

28. A process for expressing a gene, said process comprising providing a cell comprising a stably episomally replicating vector of claim 1 and cultivating said cell under suitable conditions for the expression of said gene.

29. A composition comprising a vector of claim 1 or a cell of claim 17 and further a transfection system selected from the group consisting of transfection systems consisting of a lipid, a polymer, a peptide, an a porphyrin.
